(19) United States
(12) Patent Application Publication
(10) Pub. No.: US 2010/0076023 A1
(43) Pub. Date: Mar. 25, 2010

(54) AMORPHOUS SOLID COMPOSITION CONTAINING A PYRAZOLE-3-CARBOXAMIDE IN AMORPHOUS FORM AND STABILISING CARRIERS

(75) Inventors: Jean Alié, Montpellier (FR); Jérome MENEGOTTO, Donneville (FR); Michel BAUER, Caluire et Cuire (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/543,942

(22) Filed: Aug. 19, 2009

Related U.S. Application Data
(63) Continuation of application No. PCT/FR2008/000216, filed on Feb. 20, 2008.

(30) Foreign Application Priority Data
Feb. 23, 2007 (FR) 0701377

(51) Int. Cl. A61K 31/454; C07D 401/02

(52) U.S. Cl. 514/326; 546/211

(57) ABSTRACT

The invention relates to a pyrazole-3-carboxamide in amorphous form, to an amorphous solid solution containing the same, and more generally to pharmaceutical composition containing the same

This application is a continuation of International application No. PCT/FR2008/000,216, filed Feb. 20, 2008, which is incorporated herein by reference in its entirety; which claims the benefit of priority of French Patent Application No. 07/01,377, filed Feb. 23, 2007.

The present invention relates to a pyrazole-3-carboxamide derivative in amorphous form, to an amorphous solid solution containing it, and more generally to the pharmaceutical compositions containing it. The term “amorphous form” is also intended to mean non-crystalline form.

The present invention also relates to the processes for preparing said amorphous form, said amorphous solid solution and said pharmaceutical compositions.

The term “pyrazole-3-carboxamide derivative” is intended to mean a compound selected from N-piperidino-5-(4-bromophenyl)-1-(2,4-dichlorophenyl)-4-ethylpyrazole-3-carboxamide and N-piperidino-5-(4-chlorophenyl)-1-(2,4-dichlorophenyl)-4-methylpyrazole-3-carboxamide or one of their pharmaceutically acceptable salts and/or of their solvates. In the present description, these compounds are known as “active ingredients according to the invention”.

N-piperidino-5-(4-bromophenyl)-1-(2,4-dichlorophenyl)-4-ethylpyrazole-3-carboxamide, hereinafter referred to as compound A, the international nonproprietary name of which is surinabant, is described in European patent EP-1 150 961 B1 or application WO 00/46209. The processes for preparing surinabant described in Examples 1 and 2 of EP-1 150 961 B1 or WO 00/46209 lead to crystalline products. No mention of an amorphous product is made in these documents.

N-Piperidino-5-(4-chlorophenyl)-1-(2,4-dichlorophenyl)-4-methylpyrazole-3-carboxamide, hereinafter referred to as compound B, the international nonproprietary name of which is rimonabant, is described in European patent EP 656 354 B1. The processes for preparing rimonabant described in Examples 1 and 2 of EP-656 354 B1 lead to crystalline products. No mention of an amorphous product is made in this document. Application WO 2006/021652 relates to a process for preparing rimonabant; Example 1 leads to a crystalline product. No mention of an amorphous product is made in this application.

The rimonabant and surinabant compounds are cannabinoid CB1 receptor antagonists.

These compounds are molecules which are relatively water-insoluble; their water-solubilities are respectively 0.1 μg/ml and 1 μg/l at pH=6.5. Furthermore, these compounds have high membrane permeability coefficients: respectively 78×10⁻⁷ cm/s and 96×10⁻⁷ cm/s on the Caco-2 cell model, as described by M. G. Gres et al., in Pharmaceutical Research, 1998, 15(5), 726-733.

A pharmaceutical composition containing a pyrazole-3-carboxamide derivative in micronized form and a surfactant wetting agent has been described in European patent EP-B-969 832. A pharmaceutical composition containing compound B as a mixture with Poloxamer 127 and a macrogol glyceride is described in international application WO 98/043 635.

Patent application WO 2004/009 057 describes a process for preparing a dispersion of crystalline nanoparticles in an aqueous medium and the use of surfactant at a low concentration for preventing the solubilization of said nanoparticles; and examples of preparation concerning in particular compound A and compound B.

Patent application WO 2005/046 690 describes self-emulsifiable or self-microemulsifiable pharmaceutical forms containing a pyrazole-3-carboxamide derivative for improving the solubilization of compounds A and B and their derivatives and the bioavailability in humans. These pharmaceutical forms are liquids or semi-solids.

Patent application WO 2006/087 732 describes an amorphous form of rimonabant hydrochloride.

Amorphous solid solutions containing a pyrazole-3-carboxamide derivative according to the invention in amorphous form have now been found, which have the advantage of being physically stable over a long period of time, under stressing conditions. Furthermore, these amorphous solid solutions have the advantages of being easy to handle, easy to use and easy to administer to humans. Other advantages relate to the increase in solubility of rimonabant and surinabant and the improvement in dissolution rate of rimonabant and surinabant.

The term “stressing conditions” is intended to mean in particular a temperature above 20-25° C., such as 100° C., and/or a relative humidity (RH) above 50%. The term “stressing conditions” can also relate to the conditions proposed by the International Conference on Harmonization (ICH); for example: 25° C./60% RH, 30° C./65% RH.

The present invention also relates to the pharmaceutical compositions comprising the amorphous solid solution.

The amorphous solid solutions according to the present invention comprise an amorphous homogeneous mixture of the amorphous active ingredient and of one or more amorphous excipients, in which the amorphous structure of the active ingredient is physically stabilized by one or more stabilizing excipients. Thus, the amorphous solid solutions according to the present invention are stable at ambient temperature.

The expression “amorphous active ingredient” signifies that the active ingredient, i.e. the pyrazole-3-carboxamide derivative according to the invention, contained in the amorphous solid solution is in the amorphous state, i.e. there is a minimum of 80% of active ingredient in the amorphous state in the amorphous solid solution, preferably 90% and more preferably 95% of the active ingredient, or even 100% in the amorphous state. The term “amorphous active ingredient” is also intended to mean a non-crystalline active ingredient.

Thus, a subject of the present invention is a pyrazole-3-carboxamide derivative according to the invention, in amorphous form. More particularly, the present invention relates, firstly, to the amorphous form of surinabant and, secondly, to the amorphous form of rimonabant.

The amorphous forms of surinabant and of rimonabant and also of their salts and/or of their solvates can be prepared in particular by the following processes: melt-quenching, lyophilization, milling, spray-drying (atomization), cylinder drying (drum drying), the addition of an anti-solvent (non-solvent) or by any other process for obtaining surinabant and rimonabant and also their salts and/or their solvates in the amorphous state.

Thus, according to the melt-quenching process, a crystalline form of the pyrazole-3-carboxamide derivative is heated in a closed chamber, such as an oven at a temperature above 145° C. for a period of time between 1 minute and 30 minutes, for example 10 minutes, and then rapidly cooled, for example by quenching in liquid nitrogen. The product is preferably heated at a temperature of between 145° C. and 250° C., and, for example, 180° C.

The amorphous form of rimonabant is characterized by a glass transition temperature of between 65° C. and 95° C.; the amorphous form of surinabant is characterized by a glass transition temperature of between 60° C. and 90° C.

In the anhydrous state and free of solvent, the amorphous form of rimonabant is characterized by a glass transition temperature of between 75° C. and 85° C.

In the anhydrous state and free of solvent, the amorphous form of surinabant is characterized by a glass transition temperature of between 70° C. and 80° C.

If traces of water and/or of solvent are present, the glass transition temperature may be below those indicated above for the 2 anhydrous compounds without solvent.

If structural relaxation, commonly termed physical ageing, is present, the glass transition temperature may be above those indicated above for the 2 anhydrous compounds without solvent.

The glass transition temperature can be determined by various techniques. Preferably, the glass transition temperature is determined by differential scanning calorimetric analysis (DSC). In this case, the glass transition temperature is defined by the median point of the jump in calorific capacity.

According to the technique used, the glass transition temperature may vary. The other techniques are, for example, dynamic dielectric spectroscopy (DDS) and dynamic mechanical analysis (DMA).

Another characteristic of the amorphous form of rimonabant is its X-ray diffractogram which shows the presence of a halo and the absence of diffraction peaks, characteristics indicating the absence of a crystalline phase. These characteristics for amorphous rimonabant are demonstrated on the diffractogram of FIG. 14.

Another characteristic of the amorphous form of surinabant is its X-ray diffractogram which shows the presence of a halo and the absence of diffraction peaks, characteristics indicating the absence of a crystalline phase. These characteristics for amorphous surinabant are shown on the diffractogram of FIG. 17.

Another characteristic of the amorphous form of rimonabant is the presence of a jump in calorific capacity registered by DSC. This characteristic for amorphous rimonabant is demonstrated in FIG. 13.

Another characteristic of the amorphous form of surinabant is the presence of a jump in calorific capacity registered by DSC. This characteristic for amorphous surinabant is demonstrated in FIG. 16.

The term “solid solution” is intended to mean a solid system constituted of a single phase and comprising at least two different chemical compounds, in which one compound is dispersed at the molecular scale in at least a second compound. In the present case, the term “amorphous solid solution” corresponds to a solid solution comprising the amorphous active ingredient and one or more stabilizing excipients themselves in amorphous form in the amorphous formulation.

Thus, the present invention also relates to an amorphous solid solution of a pyrazole-3-carboxamide derivative according to the invention in amorphous form with one or more stabilizing excipients.

More particularly, the present invention relates to an amorphous solid solution comprising rimonabant and/or one of its salts and/or solvates in amorphous form with one or more stabilizing excipients themselves in amorphous form.

More particularly, the present invention relates to an amorphous solid solution comprising surinabant and/or one of its salts and/or solvates in amorphous form with one or more stabilizing excipients themselves in amorphous form.

The term “stabilizing excipient” is intended to mean any excipient that is miscible at the molecular scale, with the amorphous active ingredient within the amorphous solid solution according to the invention.

Preferably, according to the present invention, the stabilizing excipients are low-molecular-weight molecules, polymers or a mixture thereof.

According to the invention, use may be made of stabilizing excipients selected from pharmaceutically acceptable acids, polyols or a polymer excipient selected from:
methacrylate copolymers,
vinyl homopolymers and copolymers,
polydextroses,
cellulosic polymers,
chemically modified starches,
pectins,
chitin derivatives,
polymers of natural origin,
polyalkylene oxides,
polyethylene glycols.

Thus, the present invention relates to an amorphous solid solution containing one or more stabilizing excipients as listed above, for example:
a polymer excipient,
several polymer excipients,
a pharmaceutically acceptable acid,
several pharmaceutically acceptable acids,
a polymer excipient and a pharmaceutically acceptable acid,
several polymer excipients and a pharmaceutically acceptable acid,
several polymer excipients and several pharmaceutically acceptable acids,
one or more polymer excipient(s) and one or more polyols(s).

Preferably, the total number of moles of stabilizing excipient(s) is at least equal to the number of moles of amorphous active ingredient. In the specific case where the stabilizing excipient is a polymer, the amount of amorphous active ingredient in the amorphous solid solution according to the present invention is such that the number of units (monomers) of the stabilizing polymer excipient is at least equal to the number of molecules of amorphous active ingredient. When the stabilizing excipient(s) is (are) a pharmaceutically acceptable acid or pharmaceutically acceptable acids comprising one or more acid functions, it is the total number of acid functions which is preferably at least equal to the number of moles of amorphous active ingredient.

The term “methacrylate copolymers” is intended to mean cationic copolymers of dimethylaminoethyl methacrylates and of neutral methacrylic esters and anionic copolymers of methacrylic acid and of methacrylic acid esters, such as, for example, the methacrylic acid/methyl methacrylate (1:1) copolymer, the methacrylic acid/methyl methacrylate (1:2) copolymer, the methacrylic acid/ethyl acrylate (1:1) copolymer or the basic butyl methacrylate copolymer. These copolymers are described in US Pharmacopeia NF21 and in the European Pharmacopoeia, 2002, Suppl. 4.4; they are sold in particular by the company Rohm under the generic name EUDRAGIT®.

The term “vinyl homopolymers and copolymers” is intended to mean polymers of N-vinylpyrrolidone, in particular povidone, copovidone and polyvinyl alcohol.

The term “polydextroses” is intended to mean polydextroses having a molecular weight of no more than 22 000 g/mol, as measured in the known manner by gel permeation chromatography (or exclusion chromatography) with a refractometric detector, more particularly having an average molecular weight of between 150 g/mol and 5000 g/mol, in particular between 1000 g/mol and 2000 g/mol. Among the polydextroses that may be used in the composition according to the invention, mention may in particular be made of the polydextroses sold by the company Pfizer under the names “polydextrose A” and “polydextrose K”, having an average molecular weight of between 1200 and 2000, and the family of polydextroses sold by the company Danisco under the name “LITESSE®”, such as “LITESSE® II”, and more particularly “LITESSE® ULTRA™ ” having an average molecular weight of between 182 and 5000.

The term “cellulosic polymers” is intended to mean alkylcelluloses, in particular methylcellulose, hydroxyalkylcelluloses, in particular hydroxymethylcellulose, hydroxyethylcellulose, hydroxypropylcellulose, hydroxybutylcellulose and weakly substituted hydroxypropylcellulose, hydroxyalkylalkylcelluloses, in particular hydroxyethylmethylcellulose and hydroxypropylmethylcellulose, carboxyalkylcelluloses, in particular carboxymethylcellulose, carboxyalkylcellulose salts, in particular sodium carboxymethylcellulose, carboxyalkylalkylcelluloses, in particular carboxymethylethylcellulose, esters of cellulose derivatives, in particular hydroxypropylmethylcellulose phthalate, hydroxypropylmethylcellulose acetate succinate, cellulose acetate phthalate-hydroxypropylcellulose such as that sold under the name KLUCEL® by the company Aqualon, hydroxyethylcellulose such as that sold under the name NATROSOL® by Aqualon and hydroxypropylmethylcellulose acetate succinate such as that sold under the name AQOAT® by the company Shin-Etsu.

The term “chemically modified starches” is intended to mean derived starches, or starches extracted from maize, from potato, from rice, from wheat or from tapioca.

The term “chitin derivatives” is intended to mean, for example, chitosan.

The term “polymers of natural origin” is intended to mean tragacanth gum, gelatin, sodium alginate, pullulan, gum arabic, guar gum, agar-agar and xanthan gum.

The term “polyalkylene oxides” is intended to mean polyethylene oxides, polypropylene oxides and copolymers of ethylene oxide and of propylene oxide.

The term “polyethylene glycols” is intended to mean preferably those having a molecular weight of greater than 1500.

The term “polyols” is intended to mean preferably sorbitol, xylitol, mannitol, erythritol and polyethylene glycols.

As stabilizing excipient, use may be made of pharmaceutically acceptable acids which have one, or even more, acid functions, such as hydrochloric acid, sulfuric acid, thiocyanic acid, L-aspartic acid, maleic acid, phosphoric acid, glutamic acid, (+)-L-tartaric acid, fumaric acid, galactaric acid, citric acid, D-glucuronic acid, glucoheptonic acid, (−)-L-malic acid, hippuric acid, D-gluconic acid, (+)-L-lactic acid, (±)-DL-lactic acid, ascorbic acid, succinic acid, glutaric acid, adipic acid, sebacic acid, acetic acid, capric acid, lauric acid, palmitic acid and stearic acid. According to the present invention, the preferred acids are citric acid and fumaric acid.

Preferably, the stabilizing excipients according to the invention are polymers which have a glass transition temperature above 75° C.

Among the stabilizing excipients having a glass transition temperature above 75° C., the following polymers are preferred:
copovidone, i.e. the “PVPVA” copolymer, namely the copolymer of N-vinylpyrrolidone and vinyl acetate, and more precisely poly(N-vinylpyrrolidone) 60%—vinyl acetate 40%, as sold under the name KOLLIDON VA 64® by the company BASF,
acrylic and methacrylic polymers, such as, for example, the basic butyl methacrylate copolymer, the methacrylic acid/methyl methacrylate (1:1) copolymer, the methacrylic acid/ethyl acrylate (1:1) copolymer, the methacrylic acid/methyl methacrylate (1:2) copolymer sold by the company Röhm under the name EUDRAGIT®, namely, respectively, EUDRAGIT® E 100, EUDRAGIT® L 100, EUDRAGIT® L 100-55 and EUDRAGIT® S100; the methacrylic acid/methyl methacrylate (1:1) copolymer (EUDRAGIT® L 100) and the methacrylic acid/ethyl acrylate (1:1) copolymer (EUDRAGIT® L 100-55) being preferred.

In the context of the invention, different types of process are used for preparing the amorphous solid solution. A first variant in which the pyrazole-3-carboxamide derivative is dissolved in at least one solvent and a second variant in which the pyrazole-3-carboxamide derivative is not dissolved in a solvent stand out in particular.

According to the first variant, the process for preparing the amorphous solid solution of the invention is characterized in that:

a) the pyrazole-3-carboxamide derivative according to the invention in amorphous form or in crystalline form and the stabilizing excipient are dissolved in an appropriate solvent in order to form a liquid solution,

b) the solvent is eliminated.

The amorphous solid solution thus obtained is in powdered form.

The term “appropriate solvent” is intended to mean a solvent or a mixture of several solvents in which the active ingredient and the stabilizing excipient are soluble, i.e. they have a solubility of greater than 1 mg/ml. A mixture of solvents is preferred if the active ingredient and the stabilizing excipient require different solvents in order to achieve the desired solubility. Examples of appropriate solvents include dioxane, dichloromethane, acetone, ethanol and water, and mixtures thereof. The preferred solvent is a mixture of water and ethanol.

The solution obtained in step a of the process is desolvated in step b by means of a process such as lyophilization, spray-drying (atomization), cylinder drying (drum drying) or the addition of a non-solvent (anti-solvent). Desolvation by cylinder drying is preferred and the solution obtained in the first step is referred to as “solution for cylinder drying”.

According to the second variant, the amorphous solid solution of the invention can be prepared according to a process, characterized in that the mixture of the pyrazole-3-carboxamide derivative in crystalline or amorphous form and of the stabilizing excipient(s) is treated either by melting and rapid cooling (melt-quenching method), or by injection molding, or by extrusion, or by any other method known to those skilled in the art.

According to the second variant, the amorphous solid solution of the invention can be prepared alternatively by another process, characterized in that the pyrazole-3-carboxamide derivative in crystalline or amorphous form and the stabilizing excipient(s) are milled together; the latter process is called co-milling.

The solid solution thus obtained by one of the processes according to the invention can be milled so as to obtain a fine powder (particle size<300 μm).

The amorphous solid solution according to the invention constitutes a homogeneous phase which can itself be associated with other excipients, without, however, these constituents modifying the physical structure of the amorphous solid solution. For this reason, the present invention also relates to pharmaceutical compositions containing the amorphous solid solution according to the invention, in particular the pharmaceutical compositions for oral administration.

Thus, according to another aspect of the invention, one or more pharmaceutically acceptable excipients may be combined with the amorphous solid solution powder so as to form a pharmaceutical composition for oral administration. Such pharmaceutically acceptable excipients may include one or more diluents such as, for example, microcrystalline cellulose, lactose, mannitol, pregelatinized starch and equivalents; one or more disintegrating agents such as, for example, sodium glycolate starch, crospovidone, sodium croscarmellose and equivalents; one or more lubricants such as, for example, magnesium stearate, sodium stearyl fumarate and equivalents; one or more sweeteners such as, for example, sucrose, saccharin and equivalents; one or more flavor enhancers such as, for example, mint, methyl salicylate, orange flavoring, lemon flavoring and equivalents; one or more dyes; preserving agents, one or more buffers; and/or any other excipients depending on the galenical form used.

The pharmaceutical compositions of the present invention preferably contain a therapeutically effective amount of the active ingredient according to the invention. The pharmaceutical compositions of the present invention may be administered, preferably orally, to patients, including but not limited to mammals such as humans, for example in the form of a hard or soft gelatin capsule, a tablet, a pill, granules or a suspension.

It is also clear to those skilled in the art that the pharmaceutical compositions of the present invention can be administered in combination with other therapeutic agents and/or prophylactic agents and/or medicaments which are not medically incompatible with one another.

Thus, the present invention relates most particularly to an amorphous pharmaceutical composition in solid form, for the oral administration of an amorphous pyrazole-3-carboxamide derivative selected from: N-piperidino-5-(4-bromophenyl)-1-(2,4-dichlorophenyl)-4-ethylpyrazole-3-carboxamide and N-piperidino-5-(4-chlorophenyl)-1-(2,4-dichlorophenyl)-4-methylpyrazole-3-carboxamide, or of one of their salts and/or solvates, in which said amorphous pyrazole-3-carboxamide derivative is physically stabilized by one or more stabilizing excipients.

The following examples illustrate the present invention without, however, limiting it.

The amorphous solid solutions and the amorphous pyrazole-3-carboxamide derivatives according to the present invention can be characterized by:
Dynamic Dielectric Spectroscopy (DDS),
X-Ray Powder Diffractometry (XRPD) and
Differential Scanning Calorimetric Analysis (DSC).

Dynamic Dielectric Spectroscopy (DDS)

Dynamic dielectric spectroscopy is used according to J. Menegotto et al., chapter 7 in “Solid State Characterization of Pharmaceuticals”, edited by Angeline and Marek Zakrzewski, Pergamon, 2006.

Before being analyzed, the samples are placed between two electrodes forming a capacitor whose material constitutes the dielectric. The general principle of dielectric spectroscopies is based on the determination of the complex impedance Z* of the capacitor. Based on this physical quantity, the complex permittivity ε* is determined according to the relationship:

$${ɛ}^{*} = \frac{1}{i ω {C}_{0} {Z}^{*}}$$

where C₀=ε₀S/e represents the capacitance of an empty capacitor of thickness e and of surface S.

The complex permittivity ε* satisfies the following equation:

ε*=ε′−iε″

where ε′ and ε″ represent respectively the real and imaginary parts of the complex permittivity.

The representation of the loss factor tan δ=ε″/ε′ as a function of the temperature and of the frequency makes it possible to localize the various dielectric characteristics of the compound studied. The dipolar relaxations intrinsic to the sample are represented in the form of peaks. They are of two types:
secondary: noted β, associated with intramolecular movements,
primary: noted α, associated with movements of groups of molecules corresponding to the dynamic glass transition of the amorphous compound.

The determination of the relaxation time for a given temperature is carried out using the Havriliak-Negami equation.

The apparatus used is a BDS 4000 dielectric spectrometer sold by NOVOCONTROL®, the sensitivity of which is of the order of 10⁻⁴ in tan δ. The accessible frequency range is between 10⁻² Hz and 10⁹ Hz. The temperature control between −160° C. and 300° C. is provided by the Quatro system from NOVOCONTROL®.

According to Menegotto et al., it is clear that the presence of a single primary relaxation mode demonstrates the presence of a single amorphous phase and thus reflects the homogeneity of the amorphous composition at the molecular scale.

X-Ray Powder Diffractometry (XRPD)

The apparatus used is a Bragg-Brentano-type DT 500 diffractometer from Siemens®. The line used is K_{α1} of copper obtained at an accelerating voltage of 30 mA-40 kV. The diffractograms are recorded for angles of between 2° and 40° at the rate of 1°.min⁻¹ in Bragg 2-theta.

Differential Scanning Calorimetric Analysis (DSC)

The apparatus used is the 2920 provided by TA Instruments or the Pyris provided by Perkin Elmer, using nonhermetic capsules. The thermograms are recorded at the rate of 10° C./min under a dry nitrogen atmosphere at a flow rate of 50 ml/min.

The DSC thermograms of Examples 7 (FIG. 19) and 9 (FIG. 13) are recorded with the Pyris from Perkin Elmer and the thermogram of Example 10 (FIG. 16) is recorded with the 2920 from TA Instruments.

### BRIEF DESCRIPTION OF THE DRAWINGS

FIGS. 1 to 18 demonstrate a certain number of characteristics of the amorphous solid solutions according to the invention and a certain number of characteristics of the amorphous pyrazole-3-carboxamide derivatives according to the invention.

FIG. 1 represents the X-Ray powder diffractogram of the solid solution prepared in Example 1.

FIG. 2 represents the temperature-dependency of the relaxation times associated with the dynamic glass transition of surinabant, of the stabilizing excipient and of the solid solution formed in Example 1.

FIG. 3 represents the X-Ray powder diffractogram of the solid solution prepared in Example 1 after 52 days at 100° C.

FIG. 4 represents the X-Ray powder diffractogram of the solid solution prepared in Example 2.

FIG. 5 represents the temperature-dependency of the relaxation times associated with the dynamic glass transition of surinabant, of the stabilizing excipient and of the solid solution formed in Example 2.

FIG. 6 represents the X-Ray powder diffractogram of the solid solution prepared in Example 2 after 52 days at 100° C.

FIG. 7 represents the X-Ray powder diffractogram of the solid solution prepared in Example 3.

FIG. 8 represents the temperature-dependency of the relaxation times associated with the dynamic glass transition of surinabant, of the stabilizing excipient and of the solid solution formed in Example 3.

FIG. 9 represents the X-Ray powder diffractogram of the solid solution prepared in Example 6.

FIG. 10 represents the temperature-dependency of the relaxation times associated with the dynamic glass transition of surinabant, of the stabilizing excipient and of the solid solution formed in Example 6.

FIG. 11 represents the temperature-dependency of the relaxation times associated with the dynamic glass transition of surinabant, of the stabilizing excipient and of the solid solution formed in Example 7.

FIG. 12 represents the temperature-dependency of the relaxation times associated with the dynamic glass transition of rimonabant, of the stabilizing excipient and of the solid solution formed in Example 8.

FIG. 13 represents the thermogram of amorphous rimonabant prepared in Example 9.

FIG. 14 represents the X-Ray powder diffractogram of amorphous rimonabant prepared in Example 9.

FIG. 15 represents the relaxation time of the modes associated with the dynamic glass transition and with the intramolecular movements of rimonabant prepared in Example 9.

FIG. 16 represents the thermogram of amorphous surinabant prepared in Example 10.

FIG. 17 represents the X-Ray powder diffractogram of amorphous rimonabant prepared in Example 10.

FIG. 18 represents the relaxation time of the modes associated with the dynamic glass transition and with the intramolecular movements of surinabant prepared in Example 10.

FIG. 19 represents the thermogram of the amorphous solid solution of surinabant and PVPPVA (70%130% by mass) prepared in Example 7.

### EXAMPLE 1

### Preparation of a Solid Solution of 50% by Mass of Surinabant and 50% by Mass of EUDRAGIT® L 100 by the Cylinder Drying Method

The preparation of the solution for cylinder drying begins with the dissolution of the surinabant in an acetone-water mixture with stirring and heating to 40° C. in order to prevent reprecipitation. The excipient is then added, still with stirring and heating. The solution is immediately made hot using a drum dryer (cylinder drying).

The composition of the solution for cylinder drying is given in Table 1.

TABLE 1
Composition of the solution

| | Mass of | Mass of | | Concentration |
|---|---|---|---|---|
| Solvent | surinabant | excipient | Volume of | of surinabant |
| used | (g) | (g) | solvent (L) | (g/l) |
| Acetone- | 60.0 | 60.0 | 1.25 | 48.0 |
| water | | | | |
| (95:5 | | | | |
| V:V) | | | | |

The operating parameters of the cylinder drying are given in Table 2.

TABLE 2
Operating parameters for cylinder drying

| Roller rotation speed | Pressure | Temperature | Feed flow rate |
|---|---|---|---|
| (rpm) | (mbar) | (° C.) | (l/h) |
| 1.6 | 150 | 80 | 7.50 |

rpm: revolutions per minute
l/h: liters per hour

The moist product recovered at the cylinder drying outlet is dried in an oven at 60° C. under 4 mbar for 24 hours.

The powder thus obtained is analyzed.

Characterization

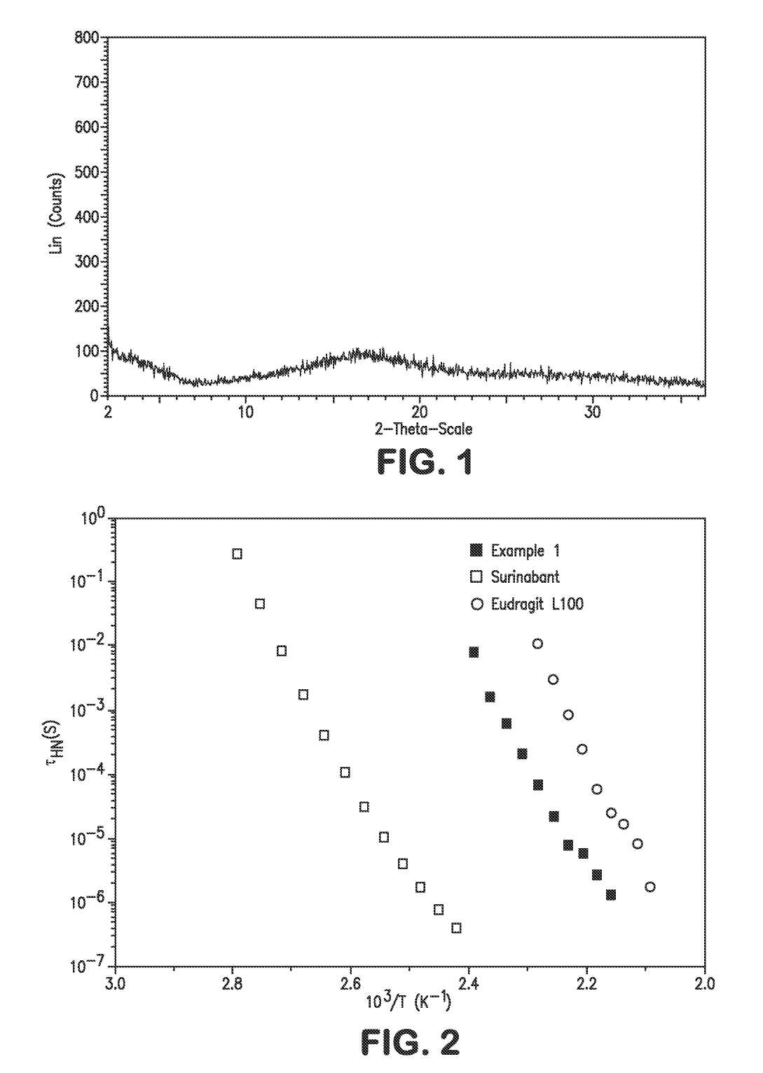

The X-Ray powder diffractogram recorded is reported in FIG. 1. The solid solution of Example 1 is amorphous as shown by the absence of diffraction peaks. This signifies that the 2 constituents present in the amorphous solid solution are amorphous.

The amorphous nature of the solid solution of powder obtained is verified by DDS. The dielectric properties of the solid solution of Example 1 are recorded as a function of the frequency (between 10⁻¹ Hz and 10⁶ Hz) in a temperature range centered around the glass transition temperatures of the various compounds. The evolution of the parameter tan δ as a function of the temperature and of the frequency reveals the presence of a single relaxation mode in the glass transition region.

FIG. 2 represents the temperature-dependency of the relaxation times, associated with the dynamic glass transition of the surinabant, of the stabilizing excipient and of the solid solution formed by mixing the two compounds.

The relaxation times associated with the solid solution of Example 1 are between those of the compound and those of the excipient. This shows that the system is homogeneous: the two compounds form an amorphous solid solution.

Stability Under Stressing Conditions

The physicochemical stability of the solid solution of powder obtained is determined at 100° C. without a controlled atmosphere, for 52 days. Several samples are placed in an oven adjusted to the temperature of 100° C. and analyzed at various times by X-ray powder diffractometry.

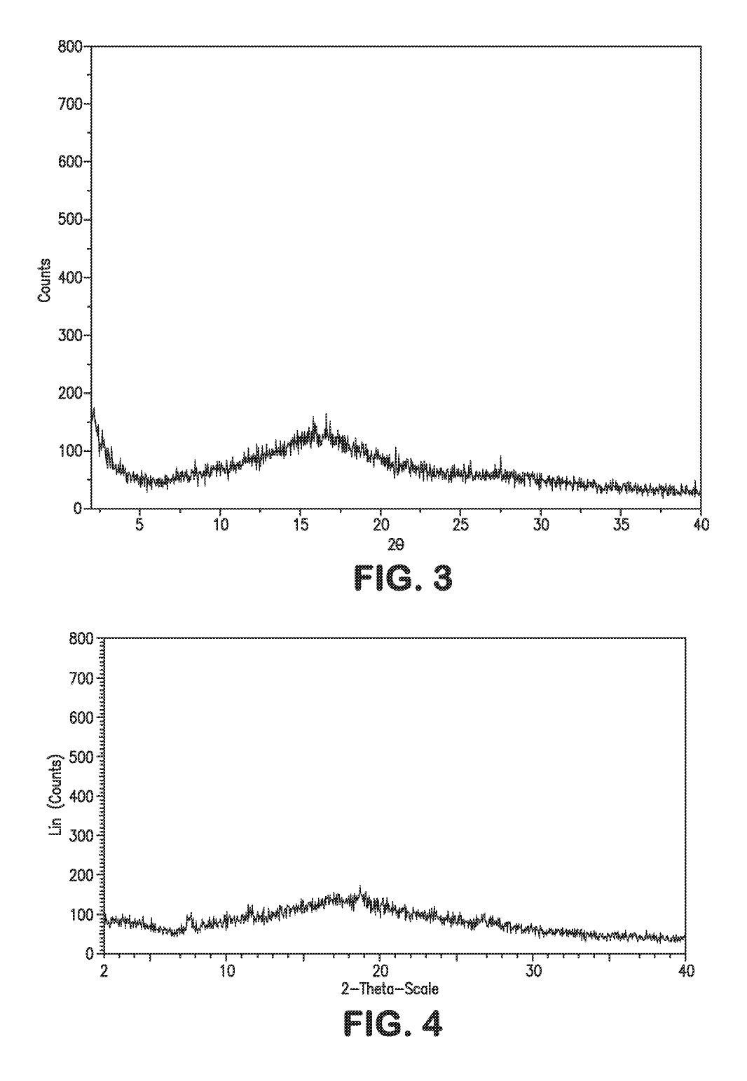

The X-Ray diffractogram of FIG. 3 shows that the solid solution of Example 1 is still amorphous after 52 days of stressing conditions at 100° C., whereas, under the same conditions, the amorphous active ingredient becomes completely crystalline in only 24 h.

### EXAMPLE 2

### Preparation of a Solid Solution of 50% by Mass of Surinabant and 50% by Mass of EUDRAGIT® L100-55 by the Cylinder Drying Method

Preparation

The preparation of the solution for cylinder drying begins with the dissolution of the surinabant in an acetone-water mixture with stirring and heating to 40° C. in order to prevent reprecipitation. The excipient is then added, still with stirring and heating, and the solution is immediately cylinder dried, under hot conditions, using a Duprat F50100 drum dryer.

The composition of the solution for cylinder drying is given in Table 3.

TABLE 3
Composition of the solution

| | Mass of | Mass of | | Concentration |
|---|---|---|---|---|
| Solvent | surinabant | EUDRAGIT | Volume of | of surinabant |
| used | (g) | L100-55 (g) | solvent (L) | (g/L) |
| Acetone- | 50.0 | 50.0 | 1.25 | 40.0 |
| water | | | | |
| (95:5 | | | | |
| V:V) | | | | |

The operating parameters of the cylinder drying are given in Table 4.

TABLE 4
Operating parameters for the cylinder drying

| Roller rotation speed | Pressure | Temperature | Feed flow rate |
|---|---|---|---|
| (rpm) | (mbar) | (° C.) | (l/h) |
| 1.6 | 150 | 80 | 8.30 |

The moist product recovered at the cylinder drying outlet is dried in an oven under the same conditions as Example 1.

The powder thus obtained is analyzed.

Characterization

The X-Ray diffractogram of the solid solution of powder obtained is reported in FIG. 4. The solid solution of Example 2 is amorphous as shown by the absence of diffraction peaks. This signifies that the 2 constituents present in the amorphous solid solution are amorphous.

The amorphous nature of the solid solution of the powder obtained is verified by DDS.

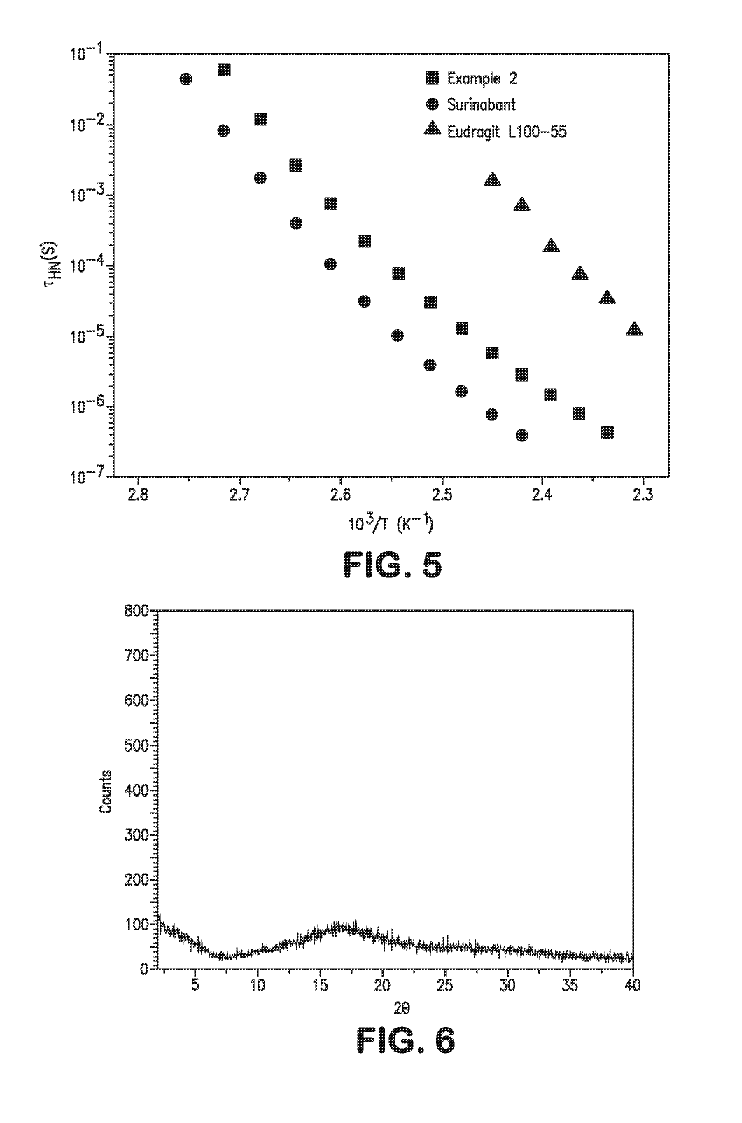

FIG. 5 represents the temperature-dependency of the relaxation times, associated with the dynamic glass transition of the compound, of the excipient and of the solid solution formed by mixing the two compounds.

The relaxation times associated with the solid solution of Example 2 are between those of the compound and those of the excipient. This shows that the system is homogeneous: the two compounds form an amorphous solid solution.

Stability Under Stressing Conditions

The physicochemical stability of the solid solution of Example 2 is determined at 100° C. without a controlled atmosphere, for 28 days. Several samples are placed in an oven adjusted to the temperature of 100° C., and are analyzed at various times by powder X-ray diffractometry.

The X-Ray diffractogram of FIG. 6 shows that the solid solution of Example 2 is still amorphous after 28 days of stressing conditions at 100° C., whereas, under the same conditions, the amorphous active ingredient becomes completely crystalline in only 24 h.

### EXAMPLE 3

### Solid Solution of Surinabant and EUDRAGIT® L100-55 Prepared by Injection-Molding and Extrusion

Preparation

A physical mixture comprising 50% by mass of EUDRAGIT® L100-55 and 50% by mass of surinabant is prepared. The physical mixing is carried out at ambient temperature (approximately 25° C.) using a TURBULA® mixer, for 30 minutes, so as to obtain a homogeneous physical mixture.

An injection press, model SPRINTER® 11 from the company Erinca, (injection molding) is fed with this mixture. The operating parameters are the following:
barrel temperature of the first heating zone: 125° C.
barrel temperature of the second heating zone: 130° C.
nozzle temperature: 140° C.
hot runner temperature: 160° C.

The mould used is such that it makes it possible to obtain a molded tablet having a size and shape substantially identical to those of a gel capsule of size 0.

The tablet thus obtained is ground and analyzed.

Characterization

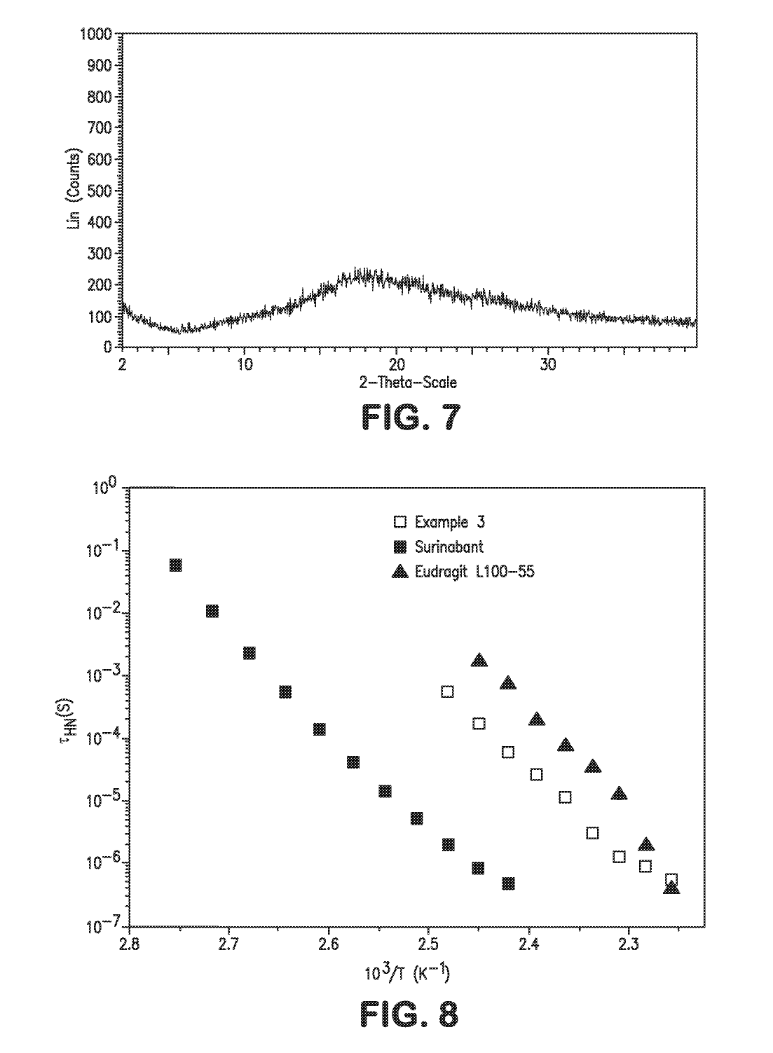

The X-Ray diffractogram of the powder obtained is recorded and reported in FIG. 7. The solid solution of Example 3 is amorphous as shown by the absence of diffraction peaks. This signifies that the 2 constituents present in the amorphous solid solution are amorphous.

The amorphous nature of the solid solution of powder is verified by DDS.

FIG. 8 represents the temperature-dependency of the relaxation times, associated with the dynamic glass transition of the compound, of the excipient and of the solid solution formed by mixing the two compounds.

The relaxation times associated with the solid solution of Example 3 are between those of the compound and those of the excipient. This shows that the system is homogeneous: the two compounds form an amorphous solid solution.

### EXAMPLE 4

### Preparation of a Solid Solution of 50% by Mass of Surinabant and 50% by Mass of EUDRAGIT® L100 by the Melt-Quenching Method

Preparation

200 mg of surinabant and 200 mg of EUDRAGIT® L100 are mixed in an agate mortar and slightly ground. This powder is deposited in a hermetic container and placed in an oven at 180° C. for 10 minutes. The container is then immersed in liquid nitrogen. The film formed at the bottom of the container is slightly ground in a mortar. The powder obtained constitutes the amorphous solid solution.

### EXAMPLE 5

### Preparation of a Solid Solution of 50% by Mass of Surinabant and 50% by Mass of EUDRAGIT® L100-55 by the Melt-Quenching Method

Preparation

200 mg of surinabant and 200 mg of EUDRAGIT® L100-55 are mixed in an agate mortar and slightly ground. This powder is deposited in a hermetic container and placed in an oven at 180° C. for 10 minutes. The container is then immersed in liquid nitrogen. The film formed at the bottom of the container is slightly ground in a mortar. The powder obtained constitutes the amorphous solid solution.

### EXAMPLE 6

### Preparation of a Solid Solution of 80% by Mass of Surinabant and 20% by Mass of Citric Acid by the Melt-Quenching Method

Preparation

160 mg of surinabant and 40 mg of citric acid are mixed in a mortar and slightly ground. This powder is deposited in a hermetic container and placed in an oven at 154° C. for 10 minutes. The container is then immersed in liquid nitrogen. The film formed at the bottom of the container is slightly ground in a mortar. The powder obtained constitutes the amorphous solid solution.

The powder thus obtained is analyzed.

Characterization

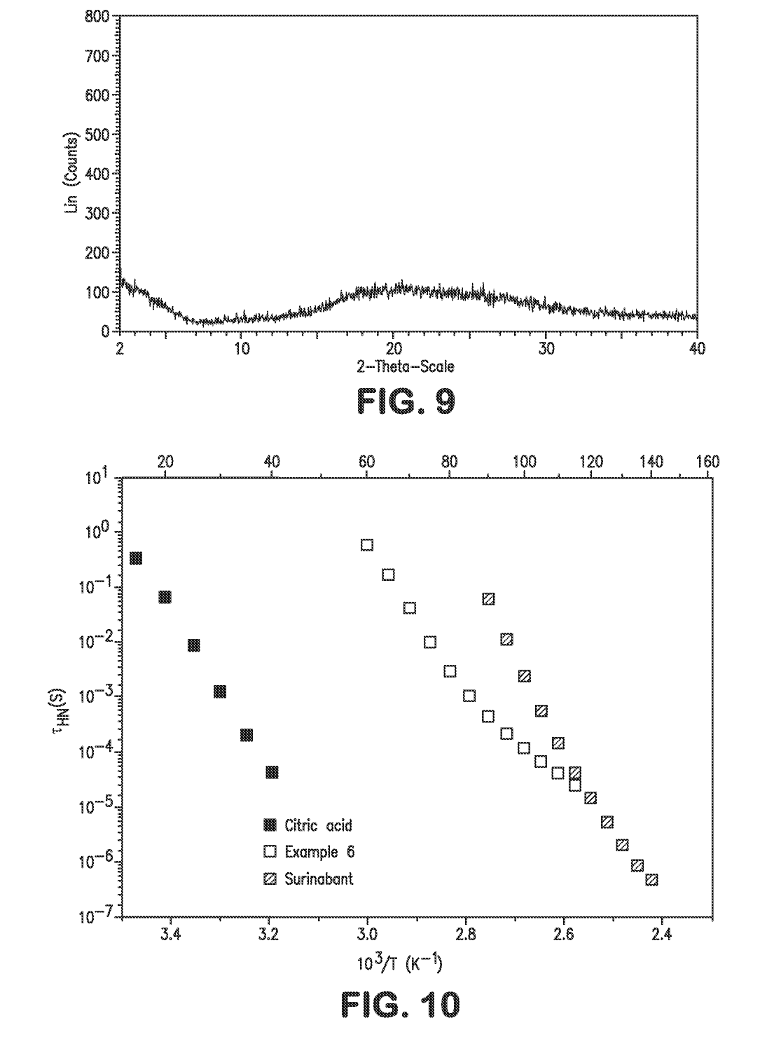

The X-Ray diffractogram of the powder obtained is recorded and reported in FIG. 9. The solid solution of the powder of Example 6 is amorphous as shown by the absence of diffraction peaks. This signifies that the 2 constituents present in the amorphous solid solution are amorphous.

The differential calorimetric analysis demonstrates a characteristic glass transition of between 40 and 70° C., and more precisely of the order of 56° C.

The amorphous solid solution nature of the powder is verified by DDS.

FIG. 10 represents the temperature-dependency of the relaxation times, associated with the dynamic glass transition of the compound, of the excipient and of the solid solution formed by mixing the two compounds.

The relaxation times associated with the solid solution of Example 6 are between those of the compound and those of the excipient. This shows that the system is homogeneous: the two compounds form an amorphous solid solution.

### EXAMPLE 7

### Preparation of a Solid Solution of 70% by Mass of Surinabant and 30% by Mass of PVPVA by the Melt-Quenching Method

Preparation

140 mg of surinabant and 60 mg of PVPVA sold under the trademark KOLLIDON VA64® are mixed in a mortar and slightly ground. This powder is deposited in a hermetic container and placed in an oven at 180° C. for 10 minutes. The container is then immersed in liquid nitrogen. The film formed at the bottom of the container is slightly ground in a mortar. The powder obtained constitutes the amorphous solid solution.

The powder thus obtained is analyzed.

Characterization

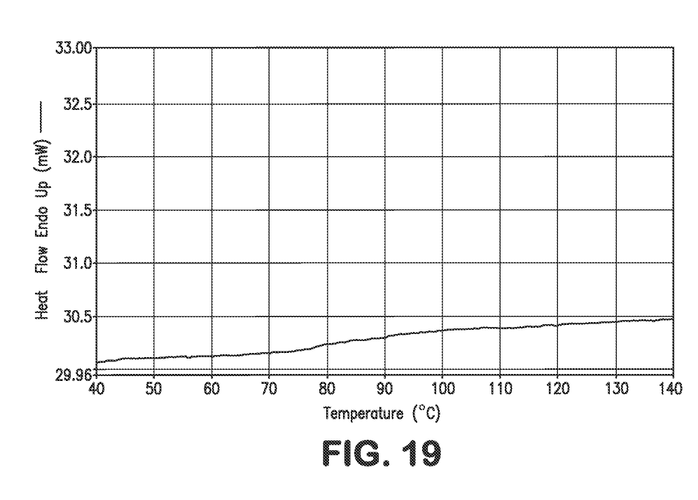

The differential scanning calorimetric analysis demonstrates a characteristic glass transition of between 68 and 98° C., and more precisely of the order of 83° C. in FIG. 19.

The amorphous nature of the solid solution of powder obtained is verified by DDS.

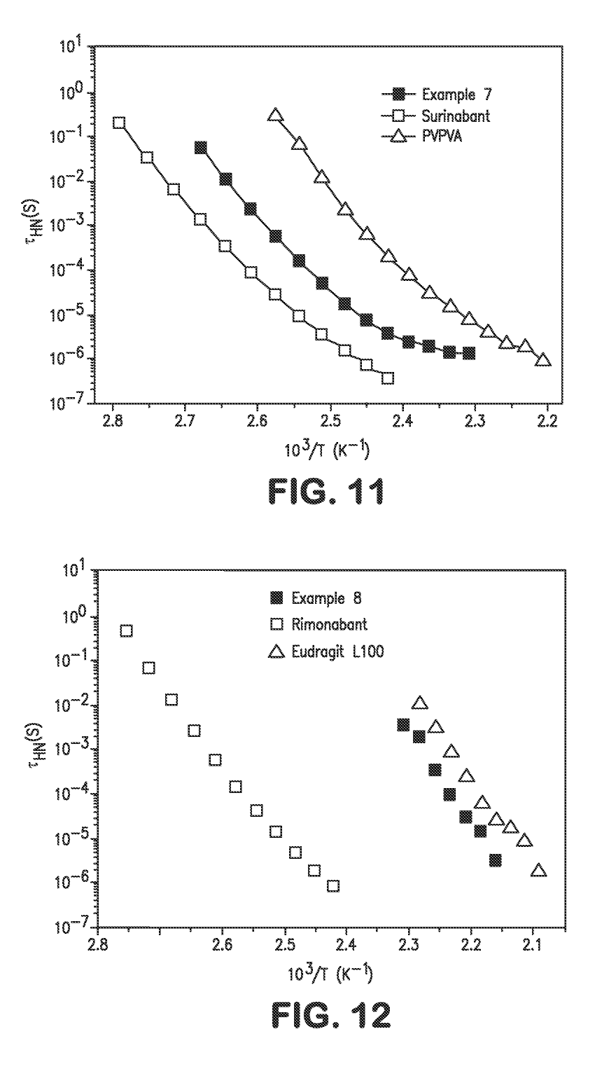

FIG. 11 represents the temperature-dependency of the relaxation times, associated with the dynamic glass transition of the compound, of the excipient and of the solid solution formed by mixing the two compounds.

The relaxation times associated with the solid solution of Example 7 are between those of the compound and those of the excipient. This shows that the system is homogeneous: the two compounds form an amorphous solid solution.

### EXAMPLE 8

### Preparation of a Solid Solution of 50% by Mass of Rimonabant and 50% by Mass of EUDRAGIT® L100 by the Melt-Quenching Method

Preparation

200 mg of rimonabant and 200 mg of EUDRAGIT® L100 are mixed in a mortar and slightly ground. This powder is deposited in a hermetic container and placed in an oven at 180° C. for 10 minutes. The container is then immersed in liquid nitrogen. The film formed at the bottom of the container is slightly ground in a mortar. The powder obtained constitutes the amorphous solid solution.

The powder thus obtained is analyzed.

Characterization

The amorphous solid solution nature of the powder is verified by DDS.

FIG. 12 represents the temperature-dependency of the relaxation times, associated with the dynamic glass transition of the compound, of the excipient and of the solid solution formed by mixing the two compounds.

The relaxation times associated with the solid solution of Example 8 are between those of the compound and those of the excipient. This shows that the system is homogeneous: the two compounds form an amorphous solid solution.

### EXAMPLE 9

### Amorphous Form of Rimonabant Prepared by Melt-Quenching

Preparation

Approximately 1 g of rimonabant is deposited in a hermetic container and placed in an oven at 180° C. for 10 minutes. The container is then immersed in liquid nitrogen. The film formed at the bottom of the container is then slightly ground in a mortar. The powder obtained constitutes the amorphous rimonabant.

The powder thus obtained is analyzed.

Characterization

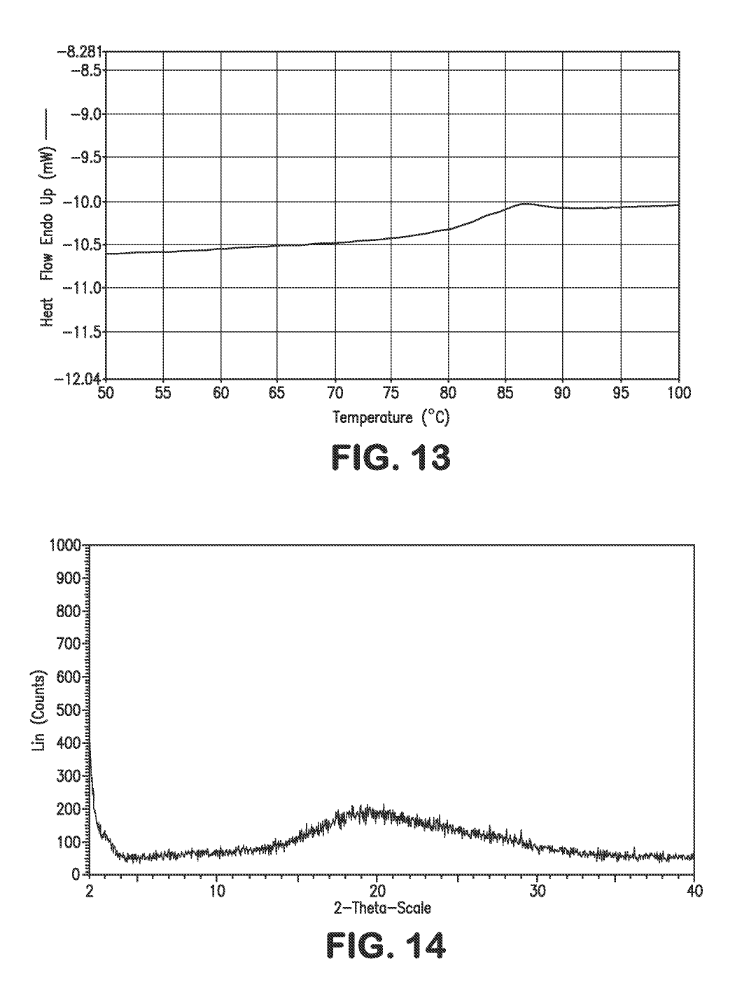

The differential scanning calorimetric analysis demonstrates a glass transition characteristic of amorphous rimonabant of between 75° C. and 95° C., and more precisely of the order of 81° C. according to FIG. 13.

The recorded X-Ray diffractogram of the powder is reported in FIG. 14. The rimonabant of Example 10 is amorphous as shown by the absence of diffraction peaks.

The dielectric properties of the powder obtained are recorded as a function of the frequency (between 10⁻¹ Hz and 10⁶ Hz) in a temperature range of between −150° C. and 130° C. The evolution of the parameter tan δ as a function of the temperature and of the frequency reveals the presence of two relaxation modes.

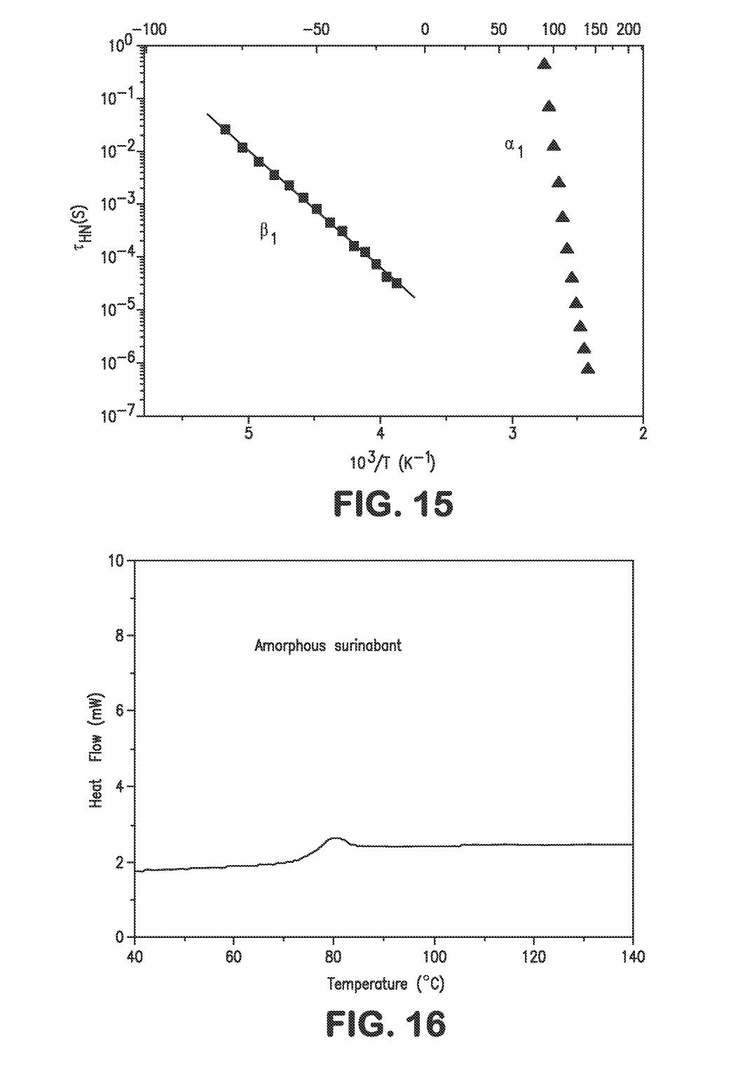

The first, at low temperatures (noted β₁), is associated with intramolecular movements. The temperature-dependency of the β₁-mode relaxation times is reported in FIG. 15. This temperature-dependency is of Arrhenius type and has an activation energy of the order of 42 kJ.mol⁻¹.

The second, in the high-temperature range (α₁), is associated with the glass transition of amorphous rimonabant. The temperature-dependency of the α₁-mode relaxation times is reported in FIG. 15. This temperature-dependency is of VTF type (Vogel-Tamman-Fulcher).

### EXAMPLE 10

### Amorphous Form of Surinabant Prepared by Melt-Quenching

Preparation

Approximately 1 g of surinabant is deposited in a hermetic container and placed in an oven at 180° C. for 10 minutes. The container is then immersed in liquid nitrogen. The film formed at the bottom of the container is then slightly ground in a mortar. The powder obtained constitutes the amorphous surinabant.

The powder thus obtained is analyzed.

Characterization

The differential calorimetric analysis demonstrates a glass transition characteristic of amorphous surinabant of between 70° C. and 90° C., and more precisely of the order of 77° C. according to FIG. 16.

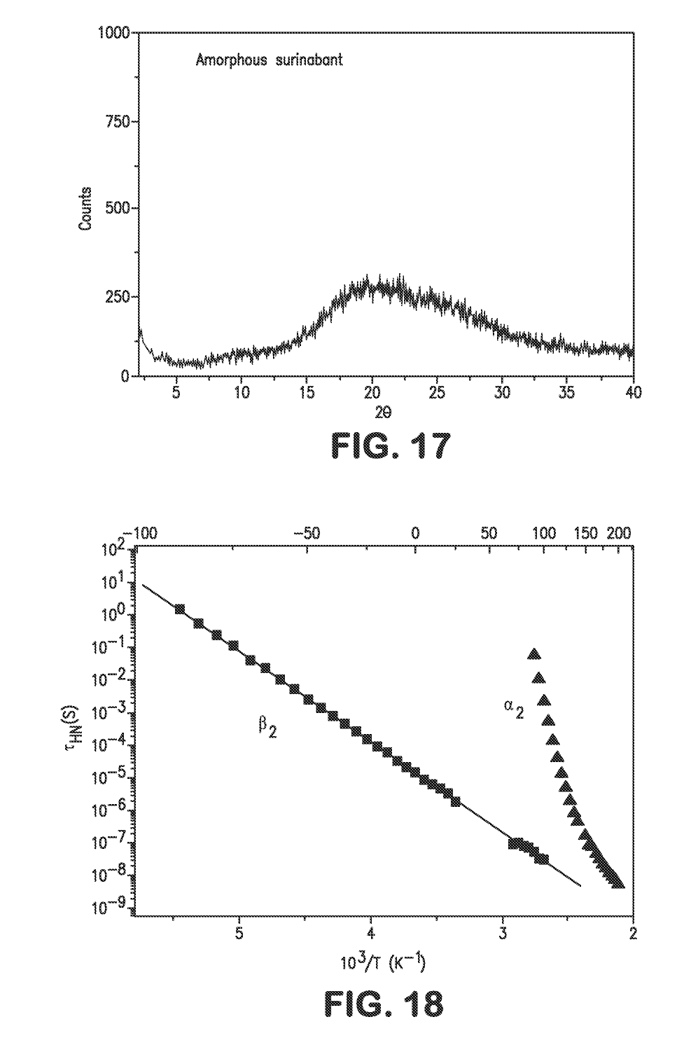

The recorded X-Ray diffractogram of the powder is reported in FIG. 17. The surinabant of Example 10 is amorphous as shown by the absence of diffraction peaks.

The dielectric properties of the amorphous powder of surinabant are recorded as a function of the frequency (between 10⁻¹ Hz and 10⁹ Hz) in a temperature range of between −160° C. and 200° C. The evolution of the parameter tan as a function of the temperature and of the frequency reveals the presence of two relaxation modes.

The first, at low temperatures (noted β₂), is associated with intramolecular movements. The temperature-dependency of the β₂-mode relaxation times is reported in FIG. 18. This temperature-dependency is of Arrhenius type and has an activation energy of the order of 53 kJ.mol⁻¹.

The second, in the high-temperature range (α₁), is associated with the glass transition of amorphous surinabant. The temperature-dependency of the α₁-mode relaxation times is reported in FIG. 18. This temperature-dependency is of VTF type.

Comparative Tests on Intrinsic Dissolution

A. Tests for evaluating the rate of intrinsic dissolution are carried out with the amorphous rimonabant obtained in Example 9 and the rimonabant in its crystalline form.

These tests are carried out under the following conditions:
Solution: Acetonitrile/H₂O (60/40)
Volume: 500 ml
Temperature: 37° C.
Rotation speed: 75 revolutions/minute
Surface area of the pellets: 0.5 cm²
Compressive force on the pellets: 2 tonnes, 20 seconds
Assay method: UV at 246 nm
Equipment: Vankel “VK700”-Kontron “Uvikon 941+”

The rates of intrinsic dissolution of the amorphous and crystalline forms of rimonabant under these conditions are 1.3 mg.min⁻¹.cm⁻² and 0.7 mg.min⁻¹.cm⁻² respectively.

These data show the advantage in terms of rate of dissolution of the amorphous form of rimonabant relative to the crystalline form of rimonabant.

B. Tests are carried out with the amorphous surinabant obtained in Example 10 and the surinabant in its crystalline form. The test conditions are identical to those of the comparative tests carried out for rimonabant (see preceding section A).

The rates of intrinsic dissolution of the amorphous and crystalline forms of surinabant under these conditions are 0.85 mg.min⁻¹.cm⁻² (mean of two determinations) and 0.2 mg.min⁻¹.cm⁻² respectively.

These data show the advantage in terms of rate of dissolution of the amorphous form of surinabant relative to the crystalline form of surinabant.

What is claimed is:

1. A pyrazole-3-carboxamide derivative in amorphous form, selected from:
N-piperidino-5-(4-bromophenyl)-1-(2,4-dichlorophenyl)-4-ethylpyrazole-3-carboxamide, which exhibits a glass transition temperature of between 60° C. and 90° C., and in that its X-ray diffractogram shows the presence of a halo and the absence of diffraction peaks; and
N-piperidino-5-(4-chlorophenyl)-1-(2,4-dichlorophenyl)-4-methylpyrazole-3-carboxamide, which exhibits a glass transition temperature of between 65° C. and 95° C., and in that its X-ray diffractogram shows the presence of a halo and the absence of diffraction peaks.

2. The pyrazole-3-carboxamide derivative according to claim 1, which is N-piperidino-5-(4-bromophenyl)-1-(2,4-dichlorophenyl)-4-ethylpyrazole-3-carboxamide.

3. The pyrazole-3-carboxamide derivative according to claim 1, which is N-piperidino-5-(4-chlorophenyl)-1-(2,4-dichlorophenyl)-4-methylpyrazole-3-carboxamide.

4. An amorphous solid solution comprising a pyrazole-3-carboxamide derivative selected from:
N-piperidino-5-(4-bromophenyl)-1-(2,4-dichlorophenyl)-4-ethylpyrazole-3-carboxamide; and
N-piperidino-5-(4-chlorophenyl)-1-(2,4-dichlorophenyl)-4-methylpyrazole-3-carboxamide;
or a salt thereof in amorphous form; and
one or more excipients themselves in amorphous form.

5. The solid solution according to claim 4, wherein the pyrazole-3-carboxamide derivative is N-piperidino-5-(4-bromophenyl)-1-(2,4-dichlorophenyl)-4-ethylpyrazole-3-carboxamide, or a salt thereof in amorphous form.

6. The solid solution according to claim 4, wherein the pyrazole-3-carboxamide derivative is N-piperidino-5-(4-chlorophenyl)-1-(2,4-dichlorophenyl)-4-methylpyrazole-3-carboxamide, or a salt thereof in amorphous form.

7. The solid solution according to claim 4, wherein the stabilizing excipient is selected from the group consisting of pharmaceutically acceptable acids, polyols and a polymer excipient.

8. The solid solution according to claim 4, wherein the stabilizing excipient is selected from the group consisting of pharmaceutically acceptable:
methacrylate copolymers,
vinyl homopolymers and copolymers,
polydextroses,
cellulosic polymers,
chemically modified starches,
pectins,
chitin derivatives,
polymers of natural origin,
polyalkylene oxides, and
polyethylene glycols.

9. The solid solution according to claim 7, wherein the stabilizing excipient is in an amount such that the total number of moles of stabilizing excipients is at least equal to the number of moles of amorphous pyrazole-3-carboxamide derivative.

10. The solid solution according to claim 4, wherein the stabilizing excipient is one or more pharmaceutically acceptable acid.

11. The solid solution according to claim 10, wherein the total number of acid functions of the pharmaceutically acceptable acid excipient is at least equal to the number of molecules of amorphous pyrazole-3-carboxamide derivative.

12. The solid solution according to claim 7, wherein one stabilizing excipient is citric acid or fumaric acid.

13. The solid solution according to claim 7, wherein the stabilizing excipient is a polyol.

14. The solid solution according to claim 7, wherein the stabilizing excipient is a polymer.

15. The solid solution according to claim 14, wherein the number of monomer unit of the stabilizing polymer excipient is at least equal to the number of moles of amorphous pyrazole-3-carboxamide derivative.

16. The solid solution according to claim 15, wherein the polymer has a glass transition temperature above 75° C.

17. The solid solution according to claim 15, wherein the polymer is selected from the group consisting of: methacrylate copolymer, vinyl homopolymer and vinyl copolymer.

18. The solid solution according to claim 17, wherein the stabilizing polymer excipient is selected from the group consisting of: the basic butyl methacrylate copolymer, the methacrylic acid/methyl methacrylate (1:1) copolymer, the methacrylic acid/methyl methacrylate (1:2) copolymer and the methacrylic acid/ethyl acrylate (1:1) copolymer.

19. The solid solution according to claim 17, wherein the stabilizing excipient is the methacrylic acid/methyl methacrylate (1:1) copolymer or the methacrylic acid/ethyl acrylate (1:1) copolymer.

20. A process for preparing the amorphous solid solution according to claim 4, comprising:
dissolving the pyrazole-3-carboxamide derivative according to claim 4 in amorphous form or in crystalline form and the stabilizing excipient in an appropriate solvent in order to form a liquid solution, and removing the solvent.

21. A process for preparing the amorphous solid solution according to claim 4, comprising:
treating a mixture of the pyrazole-3-carboxamide derivative according to claim 4 in crystalline or amorphous form and the stabilizing excipient either by melting and rapid cooling, or by injection molding, or by extrusion.

22. A process for preparing the amorphous solid solution according to claim 4, comprising:
milling together a mixture of pyrazole-3-carboxamide derivative according to claim 4 in crystalline or amorphous form and the stabilizing excipient.

23. A pharmaceutical composition comprising the amorphous solid solution according to claim 4.

24. The pharmaceutical composition according to claim 23 suitable for oral administration.
